(19) United States
(12) Patent Application Publication
(10) Pub. No.: US 2015/0360045 A1
(43) Pub. Date: Dec. 17, 2015

(54) ELECTRICAL PULSE GENERATOR TO CREATE MAGNETIC PULSES FOR THE TREATMENT OF PAIN

(71) Applicant: ZYGOOD, LLC, DAYTON, MD (US)

(72) Inventors: ROBERT E. FISCHELL, DAYTON, MD (US); WESLEY GRANT LAWSON, LAUREL, MD (US)

(21) Appl. No.: 14/338,439

(22) Filed: Jul. 23, 2014

Related U.S. Application Data
(63) Continuation-in-part of application No. 14/304,309, filed on Jun. 13, 2014.

(51) Int. Cl. A61N 2/02; A61N 2/00

(52) U.S. Cl. CPC A61N 2/02; A61N 2/008

(57) ABSTRACT

An electrical current pulse generator causes a magnetic coil attached to a human patient to create magnetic pulses within that patient's body. By Faraday's Law, the magnetic pulses cause electrical current pulses within the patient that provide pain relief with essentially zero adverse side effects. The electrical current pulse generator has dials that allow the operator to adjust several significant parameters including magnetic pulse strength, the time between pulses and the total number of pulses for a pain treatment.

This is a continuation-in-part application based upon the prior patent application entitled “Transcutaneous Magnetic Stimulation for the Treatment of Pain,” Ser. No. 14/304,309 filed with the USPTO on Jun. 13, 2014.

### FIELD OF USE

This invention is in the field of methods and devices to be used by human subjects to decrease or eliminate pain without the use of ingested or injected drugs or implanted medical devices.

### BACKGROUND OF THE INVENTION

It is well known that there are literally millions of patients throughout the world who suffer pain in various parts of their bodies. Three of the most frequent sites for pain are: (1) foot pain caused by chemotherapy or diabetic neuropathy; (2) shoulder pain often resulting from a tear or inflammation of the rotator cuff; and (3) lower back pain caused by a large variety of medical problems, particularly associated with the spine and the nerves surrounding the vertebrae. Although there are many pain medications to reduce the level of pain experienced by such patients, these drugs often are not sufficiently palliative and they typically can cause serious side effects.

One early invention to utilize magnetic fields to treat pain is described by Robert R, Holcomb in International Publication Number: WO 91/15263. The invention described in that document consists of four magnetic coils that are placed in the back of a chair or under a table where the patient's back would be exposed to the magnetic field. A DC current is placed through the four coils which results in an unchanging magnetic field to be experienced by the patient.

In U.S. Pat. No. 6,402,678, Robert E. Fischell, et al describe a device to be placed on the head which can eliminate or reduce the pain of migraine headaches by the application of an intense magnetic pulse. This device was approved by the US FDA on May 22, 2014 and is currently in use to treat patients with migraine headaches. The Fischell, et al device operates by charging capacitors to a high voltage and then discharging them into a magnetic coil to create a magnetic pulse as high as one Tesla in intensity. By Faraday's law, this changing magnetic pulse creates an electrical pulse within the brain that has been shown to eliminate migraine headaches. Because it takes about 45 seconds to charge the condensers from a battery within this Transcranial Magnetic Stimulation (TMS) device, the rate of applying magnetic pulses to the brain is extremely slow; i. e., only one pulse in about 45 seconds. Even at that, the time rate of change of the magnetic field within the brain results in an electrical current in the neurons of the brain which eliminates most migraine headaches. However, the application of more pulses per unit time could result in a more effective treatment for the relief of pain from other parts of the human body such as the feet, the shoulders or the back.

An existing system that is currently available to treat lower back pain is called TENS which is an acronym for Transcutaneous Electrical Nerve Stimulator. This device has two adhesive covered electrodes that are pasted onto the skin along the lower back. The device can then be turned on and adjusted so that the pain in the skin is acceptable while some electrical pulses enter the body in the vicinity of the spine where they can provide some relief for lower back pain. However, it would be highly advantageous to use a system that could introduce higher intensity electrical pulses into that region without causing any skin pain. That can be accomplished with TransCutaneous Magnetic Stimulation or TCMS as described herein.

### SUMMARY OF THE INVENTION

The present invention is a means and method to apply TransCutaneous Magnetic Stimulation (TCMS) to relieve pain in a human subject without the use of an analgesic drug. The TCMS system consists of a computer-controlled electrical pulse generator that would typically be plugged into a wall electrical socket and would provide repeated magnetic pulses through the skin at a site where a patient was experiencing pain. The magnetic pulses would be created by means of a pulse of electric current through a magnetic coil that has been placed in close proximity to the patient's skin at that location where the patient is experiencing pain.

For different locations on the patient's body, the magnetic coil would have a different shape. For example, for the treatment of lower back pain, a racetrack shaped coil could be placed at that region of the lower back (typically the lumbar or sacral region) where the pain is felt by the patient. The racetrack shaped coil could be attached to and placed within a specially designed belt that would place that coil in close proximity to the skin on the lower back with the buckle for the belt being located at the abdomen as is typical for belts used with male trousers. It should also be understood that pressure or inflammation of a nerve in the back can also result in pain in the buttocks and into the leg and feet. Thus, a device located on the lower back to apply TCMS in that region could also be used to reduce or even eliminate referred pain in regions of the body below the location of that nerve in the back that causes the pain in the extremities.

Another application of the present invention is a magnetic coil that is placed around the shoulder for the treatment of rotator cuff pain or any other source of shoulder pain. The unique shape for the shoulder magnetic coil would ideally be in the form of two separate coils, one on each side of the shoulder. The coil portion on each side of shoulder would cause magnetic fields that are in the same direction so as to enhance the magnetic field acting upon the tissue in the shoulder. This type of coil could also be used to wrap around the neck or the knee or any other generally cylindrical portion of a human body to reduce pain in that part of the body.

Still another application of the present invention is a coil to be used for the treatment of foot and/or ankle pain. This type of coil could be extended around the ankle to reduce pain in both the foot and the ankle. This unique design for a magnetic coil would be particularly advantageous for those patients who suffer foot and ankle pain resulting from chemotherapy or from diabetic neuropathy.

Still another application of the present invention is a flat, circular coil with an adhesive placed onto one surface so that it could be removably attached to the patient's skin at any location on a human body.

It should be understood that the electrical pulse generator would typically get its power by being plugged into a wall electrical socket. However, the use of a primary or rechargeable battery for the electrical pulse generator is certainly possible. Still further, the design described herein could obtain its electrical power from a cigar lighter electrical receptacle as located in an automobile or a truck.

The waveform for treatment would be a magnetic pulse with a rise time between 10 and 1,000 microseconds. A pulse length of approximately 200 microseconds would be ideal for this purpose. The stimulation pulse rate would optimally be at a rate between 0.1 Hz and 10 Hz with an optimum frequency being approximately 0.5 to 1.0 Hz. It is also conceived that the wave form could be approximately half of one sine wave or a square wave to optimize the relief of pain. The peak amplitude for the magnetic pulses at the patient's skin should be at least 0.1 Tesla with an optimum magnetic pulse strength at the skin being between 0.5 and 3.0 Tesla.

It should also be understood that the present invention could be used for the relief of pain that extends through a major or limited portion of a human body such as the pain of fibromyalgia, spinal stenosis, arthritis, pinched or otherwise damaged nerves, vascular inflammation, diabetic neuropathy, pain caused by chemotherapy or any other source of pain in a human body.

Thus one object of the present invention is to provide a means and method to treat pain of a human patient by the application of high intensity magnetic pulsations through the patient's skin at a location where those magnetic pulsations create subcutaneous electrical currents that result in a decrease of the patient's level of pain.

Another object of this invention is to optimize the size and shape of the magnetic coil to best fit at that specific location on the patient's body where the pain is to be treated.

Still another object of this invention is to have two magnetic coils on each side of a body part with each of the two coils enhancing the magnetic field to which that body part is exposed.

Still another object of this invention is a method to diminish the pain caused by diabetic neuropathy.

Still another object of this invention is a method to diminish the pain caused by chemotherapy.

Still another object of this invention is a method to diminish the pain caused by pressure or inflammation on the nerves in the back in the vicinity of the spine, the pain being observed in the back or below that region into the buttocks, legs and feet.

Still another object of this invention is the use of a racetrack shaped coil to be within a covering that has belt loops that can be attached around the patient's abdomen by means of a typical belt, the belt being selected to have a length ideally suited for the girth of a particular patient.

Still another object of this invention is to have an electrical current pulse generator designed to be used with a variety of shapes and sizes of magnetic coils to provide the electrical current pulses that generate the desired magnetic pulses when attached to any one of a number of different designs for magnetic coils, some of which designs are described in detail herein.

Still another object of this invention is to have an electrical current pulse generator that indicates to the operator of the generator (whether it is a medical practitioner or the patient himself/herself) as to several attributes of the treatment about to be obtained with that electrical current pulse generator.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 illustrates a magnetic coil placed around the foot and ankle of a patient for the treatment of foot and/or ankle pain, the coil being connected to an electrical pulse generator that is plugged into a wall socket.

FIG. 2 is a cross section of a magnetic coil placed around the foot and ankle of a patient for the treatment of foot pain or for the treatment of foot and ankle pain.

FIG. 3 illustrates the back of a human patient showing the back portion of the belt that is holding a magnetic coil against the patient's lower back, the coil being connected to an electrical pulse generator that is plugged into a wall socket.

FIG. 4 illustrates the front buckle and belt of a patient who is wearing the magnetic coil on his/her back for the treatment of lower back pain.

FIG. 5 is a transverse cross section of the TCMS device for the treatment of lower back pain showing a cross section of the rear portion of the belt and the magnetic coil.

FIG. 6 is a side view of a device that has two TCMS coils for the treatment of shoulder pain.

FIG. 7 is a front view of the TCMS coil system shown in FIG. 6.

FIG. 8 is a layout view of the two TCMS coils shown in FIGS. 6 and 7 and also shows the attachment of the coils to an electrical pulse generator.

FIG. 9 illustrates six different cross sections for the magnetic coil wire that can be used for the TCMS coils that are shown in FIGS. 1 through 8 inclusive.

FIG. 10 is the layout of the front panel of an advanced electrical current pulse generator that can be used with any of the magnetic coils as described herein.

### DETAILED DESCRIPTION OF THE INVENTION

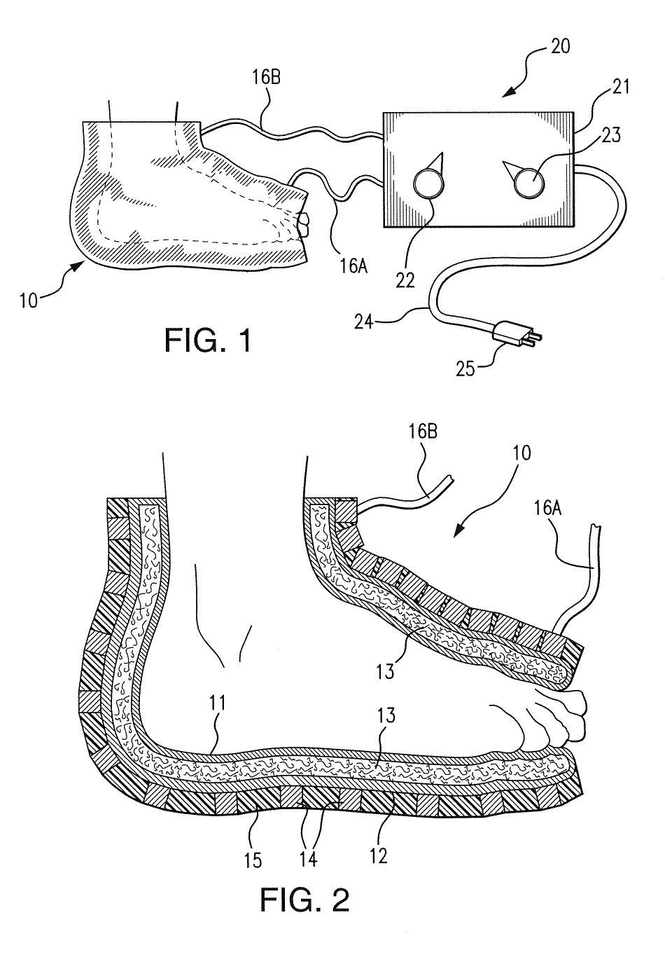

FIG. 1 illustrates a TransCutaneous Magnetic Stimulator (TCMS) magnetic coil system 10 connected to an electrical pulse generator system 20 for the treatment of foot pain. The electrical pulse generator system 20 receives its electrical power from the wire 24 that is connected to the plug 25 that would be placed into a conventional electrical socket (not shown). The electrical pulse generator system 20 is similar in design and function to the MAGSTIM 200 device that is manufactured by the MAGSTIM company in Wales, UK and sold in the USA by Jali Medical, Inc.

Although the optimum electrical pulse generator system 20 would get its power as shown in FIG. 1, it is conceived that the electrical pulse generator 21 could get its power from a primary or a rechargeable battery. The dials 22 and 23 on the face of the electrical pulse generator 21 would be used to adjust the pulse frequency and pulse amplitude for the electrical current pulses created by the electrical pulse generator system 20 to create the magnetic pulses that would be delivered by the magnetic coil system 10. These electrical current pulses would be delivered to the magnetic coil system 10 by means of the wires 16A and 16B as shown in FIG. 1. Electrical pulses with a pulse amplitude as high as 10,000 Amperes could be generated by pulse generator 21. The lowest pulse amplitude would be at least 500 Amperes to obtain the desired intensity for the magnetic pulses to be delivered by the magnetic coil system 10.

The electrical pulse generator system 20 would include conventional circuitry to generate a pulse waveform, a sinusoidal wave form, a square wave waveform or any other wave form that is found to be effective for stimulation of nerves. The frequency of the pulses could be anything between 0.1 Hz and 10 Hz with an optimum pulse rate being approximately 0.5 to 1.0 Hz. The pulse amplitude generated by the magnetic coil 10 could be anything between 0.1 Tesla and 5.0 Tesla with an optimum magnetic pulse peak intensity being between approximately 0.5 and 3.0 Tesla. When pulses are used for TCMS, the pulse rise time could be between 10 and 1,000 microseconds with an optimum pulse rise time being approximately 175±50 microseconds.

FIG. 2 is a cross section of the magnetic coil system 10 mounted onto the foot and ankle of a patient who suffers from foot pain. This foot pain would typically be caused by chemotherapy or would be a result of diabetic neuropathy. The inner lining 11 of the magnetic coil system 10 would be situated closely around the patient's foot and ankle. Surrounding the inner lining 11 would be a soft elastic type of material 13 that is contained within the inner lining 11 and the outer covering 12. It should be noted that (as shown in FIG. 2) the inner lining 11 and outer covering 12 could in fact be formed from one piece of material such as leather or a plastic material. The porous material 13 could be formed from a material such as cotton or foam rubber or any other material that would make it moderately easy for the patient to place his/her foot within the magnetic coil system 10. It is also conceived to have the patient wear a tight fitting and somewhat slippery sock when placing his/her foot into the magnetic coil system 10. That would be especially needed if the same magnetic coil system 10 is made available in a commercial medical facility for many different patients.

FIG. 2 also shows the cross section of square wire magnetic coil 14 that is wrapped around the outer covering 12. A total of 12½ turns of the magnetic coil 14 are shown in FIG. 2. Any number of turns between 4 and 30 could be used for such a magnetic coil 14 with an optimum number of turns being approximately 15±8 turns. It should also be understood that the wire of the coil 14 would be insulated on its exterior and would have a cross section that could be of any type as shown in FIG. 9.

Surrounding the magnetic coil 14 would be a coil covering member 15 that could be formed from a plastic material or from certain cloth materials. It should also be understood that the wires 14 could be adhesively attached to the outer layer 12 or there could be a plastic fill material situated between each turn of the wires of the magnetic coil 14. FIG. 2 also shows the wires 16A and 1B which are attached to the electrical pulse generator 21 as shown in FIG. 1.

The present invention conceives of the magnetic coil system 10 being made available to the patient in at least three different shoe sizes with as many as 10 different shoe sizes being envisaged as being possible for this product.

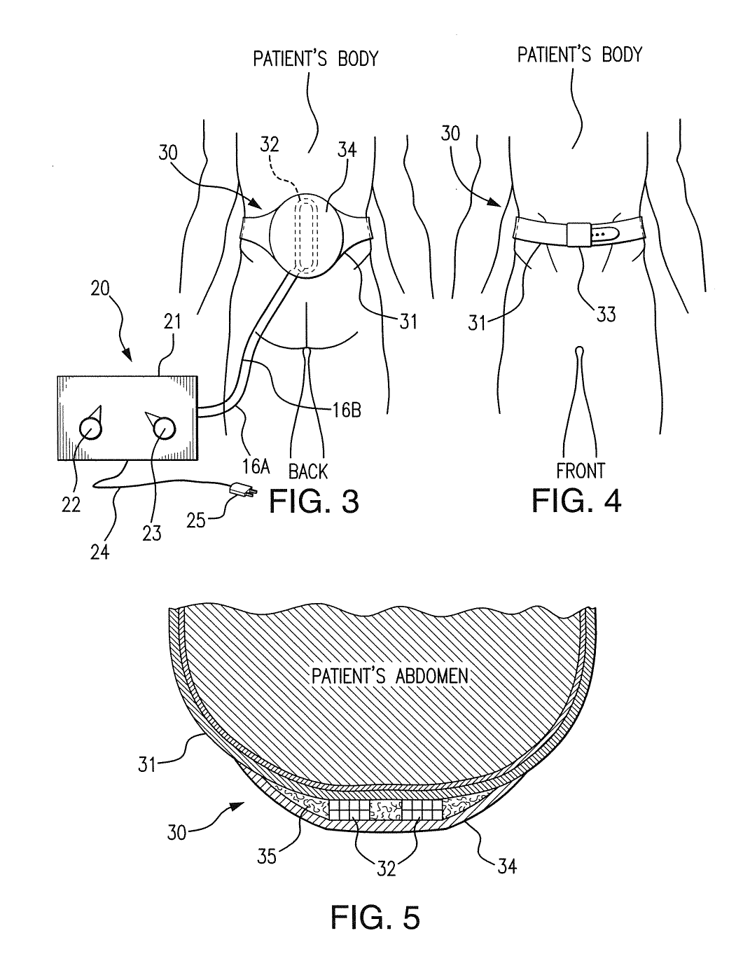

FIGS. 3, 4 and 5 illustrate a TCMS magnetic coil system 30 that is designed to eliminate or at least decrease lower back pain for patients who suffer from that all too common affliction.

FIG. 3 shows the electrical pulse generator system 20 that has an electrical pulse generator 21 attached by the wires 16A and 16B to the magnetic coil 32 that is attached to the belt 31. As can be seen in FIGS. 3 and 5, the magnetic coil 32 is in the general shape of a racetrack with multiple turns of a conducting wire that has a square cross section. Two layers of the coil 32 are shown in FIG. 5, but anywhere from one to five layers of wires could be used to accommodate approximately 15±8 turns of wire. The wire cross section could be of any type as illustrated in FIG. 9.

An important aspect of the present invention is the design of the back of the magnetic coil system 30 as shown in FIGS. 3 and 5. The outer belt cover 34 has a generally elliptical shape as it covers the magnetic coil 32 and it also has a filler material 35 (as seen in FIG. 5) having the consistency of foam rubber or an equivalent spongy material.

FIG. 4 shows the front of the patient's body showing the belt 31 that has a conventional width that is secured around the patient's body with a conventional buckle 33.

It should be understood that the design of the TCMS system 30 to treat lower back pain allows the system 30 to be applied while a patient is working at his/her desk or even if the patient is lying in bed. Thus the pain relief can be applied at most times when it would be truly convenient for such a patient to utilize such a device.

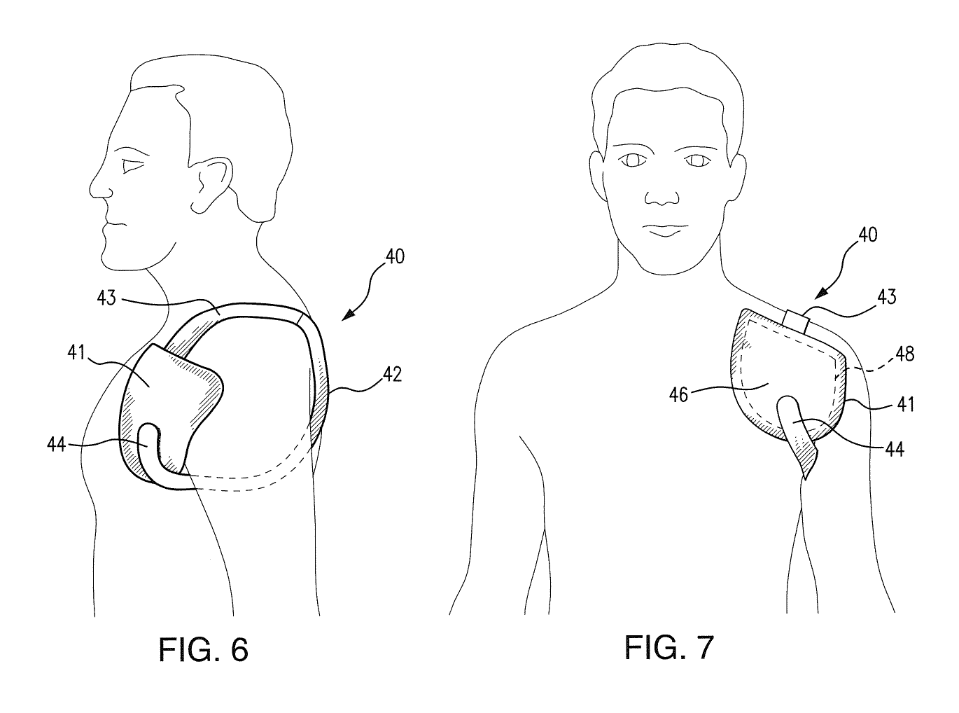

FIGS. 6, 7 and 8 illustrate a type of magnetic coil system 40 that can be used to treat shoulder pain such as that caused by an inflammation of the rotator cuff in that patient's shoulder.

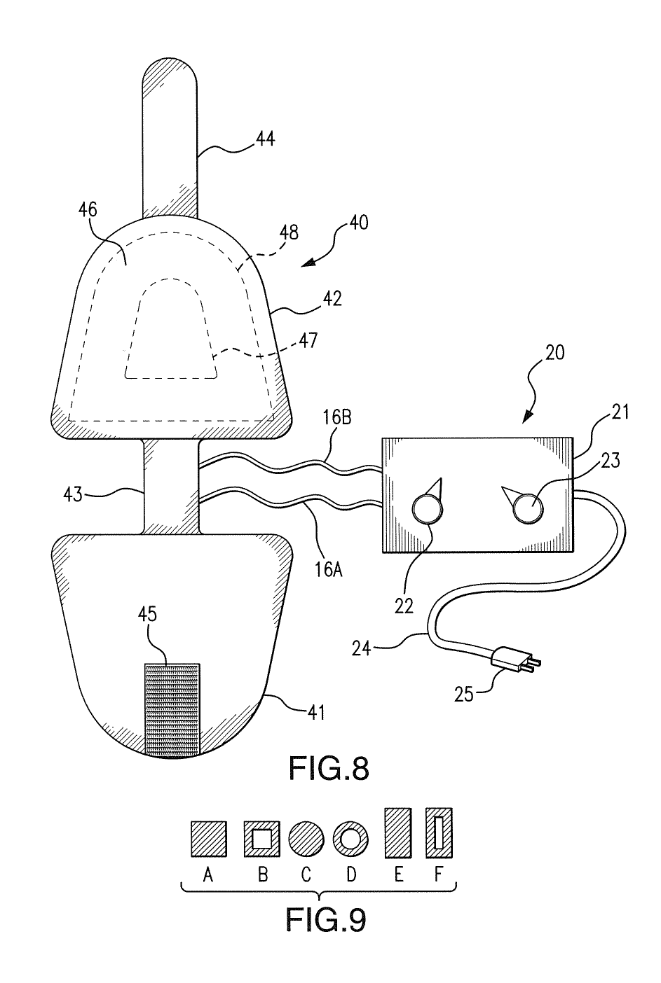

FIG. 6 is a side view of the magnetic coil system 40 that has a front coil system 41, a rear coil system 42, each of which coil system includes a magnetic coil covered by some cloth or plastic material. The magnetic coil system 40 also includes a flexible connecting member 43 and a Velcro attachment tab 44. FIG. 7 shows a front view of the magnetic coil system 40 having front coil 41 into which is shown the outer outline 48 of the magnetic coil 46, a flexible connecting member 43 and the Velcro attachment tab 44. Placing this magnetic coil system 40 onto the shoulder would be accomplished by putting the front coil 41 on his/her shoulder in front with the rear coil 42 in back with the flexible connecting member 43 being placed on top of the shoulder. The Velcro attachment tab 44 would then be brought around from the rear coil 42, under the armpit and then it would be attached to the Velcro surface 45 (as shown in FIG. 8) for securely holding the magnetic coil system 40 onto the patient's shoulder.

FIG. 8 is a layout view of the magnetic coil system 40 as used for decreasing pain at a patient's shoulder. Shown in FIG. 8 is the front coil 41, the rear coil 42, the flexible connecting member 43 and the Velcro attachment tab 44 that is designed to be placed through a patient's armpit for attachment to the Velcro attachment surface 45 that is placed on the outer surface of the front coil 41. Also shown in FIG. 8 is the inner outline 47 and the outer outline 48 of the magnetic coil 46. A similar magnetic coil (not shown) would be placed in the front coil 41. These two magnetic coils would be similar to those shown in FIG. 2 and they would be placed within a cloth or plastic cover.

FIG. 8 also shows the wires 16A and 16B that connect the magnetic coil system 40 to the electrical pulse generator 21. The electrical pulse generator system 20 includes the electrical pulse generator 21 having electric current pulse amplitude and frequency control knobs 22 and 23 and a wire 24 having a plug 25 that connects into a conventional wall socket (not shown).

FIG. 9 illustrates the cross section of various wires that could be used for the magnetic coils in any of the designs as described herein. Wire A is a typical square wire that could have a dimension of anything from 0.1×0.1 inch to 0.5×0.5 inch. Wire B could have the same outer dimensions as wire A, but would have a wall thickness between 0.01 inch and 0.1 inch. Wire C is a typical round cross section wire that could be any size from 0 AWG to 10 AWG. Wire D is a hollow wire whose outer cross section is circular. The wall thickness range for Wire D is the same as for Wire B. Wire E has a rectangular cross section with a width between 0.05 inch to 0.3 inch and a height that could lie between 0.1 inch and 0.5 inch. Wire F would have the same external dimensions as Wire E, with a wall thickness similar to Wire B. Not shown in FIG. 9 is the insulation that would always be placed on the exterior surface of any such wire.

It should be understood that any of the hollow wires shown in FIG. 9 could be used to have either gas or a liquid flow through such a wire in order to either heat or cool the magnetic coil. Some patients may be more comfortable with a magnetic coil that would cool the body part feeling pain and some patients may feel less pain if heating was used to warm the magnetic coil to improve the patient's comfort.

A useful and novel attribute of the system shown in FIG. 8 is that it could be readily adapted for placement around any essentially cylindrical type of surface of a human body. In that case, the dimensions of the front coil 41 and the rear coil 42 would be such as to exactly go around such a cylindrical surface. For example, the system shown in FIG. 8 could be used to wrap the electrical pulse generator system 40 around a knee for knee pain, the neck for neck pain, around any arm or leg for treatment of pain in those regions, or any other generally cylindrical surfaces of a human body. The present invention envisages that the outer shape of the front coil 41 and the rear coil 42 could be made to be square or round or any other shape that would be ideal for placement around any part of a human body.

Although the electrical pulse generator system 20 as described herein is always shown to have knobs 22 and 23 to adjust the intensity and frequency of the electrical current pulses emanating from that device, it should be understood that it could also have fixed values of frequency and pulse amplitude so that adjustment knobs would not be needed. It should also be understood that the magnetic pulse generator 21 could have an electrical switch to turn it to ON or OFF or it could turn on automatically when plugged into a wall socket.

A patient who has significant pain in at least one of the regions described herein may very well wish to transport his TCMS system from home to work and back or to take each system on travel. To that end, it is envisaged that each of the magnetic coil systems 10, 30 and 40 as described herein, and also the magnetic coil system 20 could have a special carrying case that has wheels and a pull-up handle as is commonly used for suitcases. The electrical pulse generator system 20 may also be designed with wheels and a pull-up handle so as to make moving it from one place to the other less difficult.

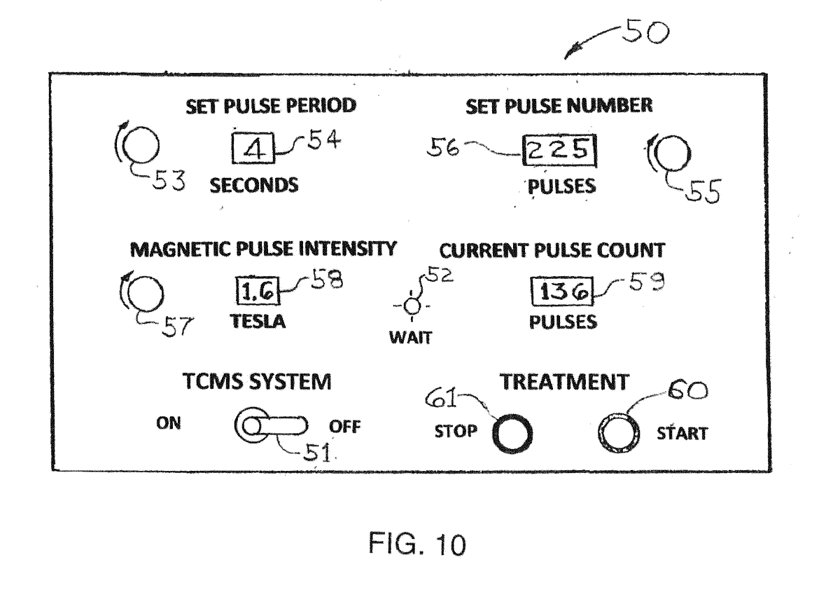

FIG. 10 illustrates the face plate of an electrical current pulse generator 50 that has unique and superior characteristics as compared to the pulse generator 21 as previously described herein. The electrical pulse generator 21, as described herein, allows the operator of this equipment to control only the amplitude and/or the frequency of the electrical pulses emitted by the electrical pulse generator 21 and does not indicate to the operator what the values are of those parameters that are being adjusted. The electrical current pulse generator 50, as described herein, can also control the amplitude and frequency of the electrical pulses generated by its electronics to power a variety of magnetic coil systems. However, the electrical current pulse generator 50 also provides several additional novel and useful features to enhance the operation of the complete system for the control of human pain.

The electrical current pulse generator 50 of FIG. 10 has an ON-OFF switch 51 that is used by the operator to turn the electrical current pulse generator 50 on or off. When not being used, the switch 51 would typically be in the OFF position as shown in FIG. 10. When the switch 51 is turned to the ON position, two things happen, namely: 1) power is provided to all the electronics of the electrical current pulse generator 50; and 2) the WAIT light 52 turns to on (or still better to flashing on and off) preferably with an amber caution light from an LED. When all the electronics of the electrical current pulse generator 50 is warmed up, the WAIT light 52 turns off, and all the indicator boxes that show a particular measurement (namely; indicators 54, 56, and 58) come on with either there last number that was in that indicator box or each number could be zero. After the WAIT light 52 goes off, the electrical current pulse generator 50 is all warmed up and ready to provide electrical current pulses to create magnetic pulses using any one of the several magnetic coils as described herein. At that time, the number in the indicator box 59 goes to zero.

When the WAIT light 52 has gone to its off state, the operator can then: 1) rotate the knob 53 to set the number of seconds between each pulse as indicted in the box 54; 2) rotate the knob 55 to set the total number of pulses that the operator feels will be sufficient to ease the pain of the patient to be treated as indicated in box 56; and 3) rotate the knob 57 to adjust the amplitude of the magnetic pulses that are expected to be delivered just below the patient's skin as indicated in box 58. After the operator feels that each of the settings are optimum for a particular patient and for a particular magnetic coil design, then the operator can press the button 60 (which turns on a green light surrounding that button) to start the treatment with a series of magnetic pulses into the patient's tissues. Using the settings as shown in FIG. 10, the patient would receive 225 magnetic pulses at an interval of 4 seconds between each of the pulses and the peak magnetic pulse intensity just below the patient's skin would be approximately 1.6 Tesla. The number in the box 59 would indicate to the operator how many pulses (136 at this instant) that have been delivered into the patient. When the treatment is concluded, the numbers in the boxes 56 and 59 would be identical. If at any time during this treatment, it was desired to stop the treatment, the operator could press the STOP button 61 and the treatment would be stopped. It should be understood that the operator could be either a health care professional or the patient herself or himself.

There are many advantages to this type of design for a generator that is used to create electrical current pulses that enter magnetic coils to create magnetic pulses into a patient's body. It is anticipated that some patients would be more comfortable and gain more pain relief if the time between pulses was either less than or more than 4 seconds. Thus, the operator could use any number of seconds for the treatment between approximately 1.0 second and (let us say) 10 seconds. Later research might even show that pulse periods either less than one second or greater than 10 seconds may be beneficial for some pain patients.

In a similar manner, it might be shown that more or less than 225 pulses would be optimum for some patients for some levels of pain in different parts of a human body. It is also possible that magnetic pulse peak amplitudes greater than or less than 1.6 Tesla may be advantageous for some patients.

The advantages of the novel features of the electrical current pulse generator 50 as described herein are that it teaches that each of these most significant parameters can be adjusted to optimize pain relief for different patients and for different body locations. The settings shown in FIG. 10 indicate that this treatment would have a peak magnetic pulse intensity of 1.6 Tesla at about 1.0 cm from the surface of the coil placed against the patient's body. Although the knob 57 could be used to lower the highest magnetic field strength that this system could deliver, it is actually unlikely that lower levels of magnetic field strength would improve treatment outcomes. Since there is no adverse side effects from even the highest level of magnetic field strength that the electrical current pulse generator 50 could deliver, it is unlikely that lower magnetic pulse intensities would be useful. However, some patients may be able to have an effective treatment that is accomplished in less time by using either a shorter time between pulses or a decreased number of pulses. For the settings shown in FIG. 10, with 225 pulses having a time between pulses of 4 seconds, this would provide for a treatment that lasts 15 minutes before the treatment has been completed and the green light of the START button 60 goes off. For some patients, an effective treatment in a shorter period of time could provide a satisfactory result for pain reduction or elimination. Other patients may benefit from a longer time between each pulse and/or a higher number of pulses. It is expected that the selection of pulse period and pulse number can only be determined by trial and error with each specific patient and for each location of the pain in the human body. A unique advantage of the electrical current pulse generator 50 of FIG. 10 is that it can be readily adjusted to optimize the treatment for pain for each pain location and for each different patient by the adjustment of these various parameters.

Another significant advantage of the design of the face plate for the electrical current pulse generator 50 shown in FIG. 10 has to do with its use in countries where languages other than English are required by law to be used for any medical device that is approved for treatments in that particular country. Specifically, essentially all non-English speaking countries in Europe and Asia require that any medical device used in that country be labeled with the language of that country. The face plate of the electrical current pulse generator 50 of FIG. 10 can have all words placed on that front plate with either printing or a clear plastic sheet pasted onto the front plate that has the word or symbol for that feature in the language of the country where it will be used. Thus the word “TREATMENT” could be written in German or the Chinese character for the word “TREATMENT” could be placed to replace the English word “TREATMENT” shown in FIG. 12. However, the lights for the START button 60 or the STOP button 61 would be respectively in green and red for all countries as would the amber flashing light for the WAIT button 52. Still further, each of the boxes 54, 56, 58 and 59 would have a number controlled by a computer system within the electrical current pulse generator 50 that provides a number as is used by essentially all nations on this planet. In this way, the need to reprogram the software for different languages for the front plate display would be avoided. This is a novel and useful advantage for the design of any medical device that is to be used in many counties having different languages.

Although the use of the electrical current pulse generator 50 as described herein consistently discusses its use for human patients, it should be understood that this equipment can also be useful to reduce the pain suffered by animals such as dogs, cats or horses or any other animal that has pain. To that end, wherever herein the word “patient” is used, that patient could also be considered to be any animal that is suffering from a condition of pain.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. An electrical current pulse generator to be used with a magnetic coil placed upon some region of a human patient for eliminating or reducing pain, the system including:
a magnetic coil system including an electrically conducting coil of wire that is adapted for placement onto a particular portion of the patient's body; and
an electrical pulse generator that has control knobs to adjust at the least the following parameters: the total number of electrical pulses to be delivered to the magnetic coil and the time between each pulse.

2. The system of claim 1 where the electrical pulse generator includes controls to adjust the amplitude of the electrical current pulses produced by the electrical pulse generator with the amplitude being expressed as the approximate number of Tesla of magnetic field intensity that is to be applied just below the patient's skin.

3. The system of claim 1 where the electrical pulses from the electrical current pulse generator have a rise time between 10 and 1,000 microseconds.

4. The system of claim 1 where the electrical pulses from the electrical current pulse generator have a rise time of approximately 175±50 microseconds.

5. The system of claim 1 where the peak intensity of the magnetic pulses created by the magnetic coil system is between approximately 0.5 and 3.0 Tesla.

6. The system of claim 1 where the peak electric current pulse emanating from the electrical pulse generator is between approximately 500 and 10,000 Amperes.

7. The system of claim 1 where each label on the front plate of the electrical current pulse generator is written in the specific language of the country in which that device will be used.

8. The system of claim 1 where each number in a number box as indicated on the front plate of the electrical current pulse generator can be set for: 1) the control of the magnetic pulse intensity; 2) the period of time between each pulse; and/or 3) the total number of pulses to be delivered into the patient for a particular treatment, where each value of these treatment parameters is controlled and set by a separate knob.

9. The system of claim 8 where the maximum number of pulses to be set by its associated control knob is 999.

10. The system of claim 1 where the maximum time that can be set by its associated control knob for the period between pulses is 99 seconds.

11. The system of claim 1 where the magnetic coil would have approximately 15±8 turns of the electrically conducting, insulated wire.

12. The system of claim 1 where the face plate of the electrical current pulse generator includes a WAIT light that remains in an on condition as long as the electronics of the electrical current pulse generator is warming up.

13. The system of claim 12 where the WAIT light is a flashing amber LED light.

14. The system of claim 1 where the electrical current pulse generator includes a START button that is designed to be pushed in order for the patient's treatment with magnetic pulses is to begin.

15. The system of claim 14 where there is a green light that illuminates in close proximity to the START button when the electrical current pulse generator is turned to its on condition for delivering pulses.

16. The system of claim 1 including a STOP button that can be pressed to stop the creation of electrical pulses by the electrical current pulse generator.

17. The system of claim 16 where a red light associated with the STOP button turned to on is located in close proximity to the STOP button.

18. A method to reduce the pain of a patient by the application of intense magnetic pulses onto that region where the patient is feeling pain, the method having the following steps:
a) creating an electrical current pulse generator system that can produce electrical current pulses having a magnitude that is between 500 and 10,000 Amperes:
b) connecting the electrical pulse generator system to a magnetic coil system placed onto that region of the body where the patient is feeling pain;
c) turning the electrical current pulse generator to its ON condition;
d) waiting for a WAIT light to go to an off state;
e) adjusting the parameters of magnetic pulse period and the number of pulses to be delivered for a specific treatment for the patient; and
f) pressing a START button to begin the treatment for pain.

19. The method of claim 18 including the step of adjusting the level of peak magnetic pulse intensity to be used during the treatment for pain.

20. The method of claim 18 including the step of turning the electrical current pulse generator to its OFF condition after the treatment for pain is completed.
